(19) United States
(12) Patent Application Publication
(10) Pub. No.: US 2022/0322992 A1
(43) Pub. Date: Oct. 13, 2022

(54) BIO-SIGNAL MEASURING APPARATUS FOR DETECTING SIGNAL PEAKS, METHOD OF DETECTING SIGNAL PEAKS IN ELECTROCARDIOGRAM, AND COMPUTER PROGRAM FOR PERFORMING METHODS

(71) Applicant: ATSENS CO., LTD., Gyeonggi-do (KR)

(72) Inventors: Kab Mun CHA, Seoul (KR); Jong Ook JEONG, Gyeonggi-do (KR)

(73) Assignee: ATSENS CO., LTD., Gyeonggi-do (KR)

(21) Appl. No.: 17/226,680

(22) Filed: Apr. 9, 2021

(51) Int. Cl. A61B 5/352; A61B 5/30; A61B 5/35

(52) U.S. Cl. CPC A61B 5/352; A61B 5/30; A61B 5/35

(57) ABSTRACT

A bio-signal measuring apparatus for detecting signal peaks includes a bio-signal sensing circuit configured to sense an electrocardiogram signal by using an electrode attached to the body. The bio-signal measuring apparatus further includes a first filtering unit, a second filtering unit, a candidate peak detector, a peak integrator, and a valid peak detector. The peak integrator is configured to define windows by time intervals and calculate, as average peak time values, average values of time values of the first candidate peaks and the second candidate peaks included in the windows. The valid peak detector is configured to calculate complexity values for time intervals including the average peak time values, determine a peak occurrence time value on the basis of the complexity value, and detect valid peaks in the electrocardiogram signal by using the peak occurrence time value.

### BACKGROUND

### 1. Field

One or more embodiments relate to a bio-signal measuring apparatus for detecting signal peaks, a method of detecting signal peaks in an electrocardiogram, and a computer program for performing the methods, where the signal peaks are detected by using a complexity value of a time interval.

### 2. Description of the Related Art

An existing stand-alone or fixed electrocardiogram measuring apparatus includes attachable electrodes having a large gap (distance) therebetween and measures an electrocardiogram signal of a user while the user maintains a fixed posture. Thus, a change in a baseline of the measured electrocardiogram signal is not great and is stable, and the magnitude of the measured electrocardiogram signal is also large.

In contrast, a wearable patch-type electrocardiogram measuring apparatus includes electrodes having a short distance therebetween and frequently causes movement of a user. In an electrocardiogram signal measured in the above way, various types of noise are generated due to the occurrence of movement in addition to general human body noise. Also, a baseline of the electrocardiogram signal measured in the above way is not stable and significantly changes. In addition, the magnitude of the electrocardiogram signal measured in the above way is also relatively less than the magnitude of an electrocardiogram signal measured by a high-priced stand-alone or fixed electrocardiogram measuring apparatus.

Accordingly, it is difficult to detect an R-peak of an electrocardiogram signal by using a patch-type electrocardiogram measuring apparatus.

### SUMMARY

One or more embodiments include a bio-signal measuring apparatus for detecting signal peaks by using a complexity value of a signal around a peak, a method of detecting signal peaks in an electrocardiogram, and a computer program for performing the methods.

One or more embodiments include a bio-signal measuring apparatus for detecting, via machine learning or an artificial intelligence algorithm, signal peaks in a time interval having a complexity value exceeding a determined threshold complexity level, a method of detecting signal peaks in an electrocardiogram, and a computer program for performing the methods.

Additional aspects will be set forth in part in the description which follows and, in part, will be apparent from the description, or may be learned by practice of the presented embodiments of the disclosure.

According to one or more embodiments, a bio-signal measuring apparatus for detecting signal peaks includes: a bio-signal sensing circuit configured to sense an electrocardiogram signal by using an electrode attached to the body; a first filtering unit configured to filter the electrocardiogram signal into a first frequency band; a second filtering unit configured to filter the electrocardiogram signal into a second frequency band; a candidate peak detector configured to detect first candidate peaks in a first filtering signal filtered by the first filtering unit and detect second candidate peaks in a second filtering signal filtered by the second filtering unit; a peak integrator configured to define windows by time intervals and calculate, as average peak time values, average values of time values of the first candidate peaks and the second candidate peaks included in the windows; and a valid peak detector configured to calculate, in the first filtering signal and the second filtering signal, complexity values for time intervals including the average peak time values, determine a peak occurrence time value on the basis of the complexity value in the first filtering signal and the complexity value in the second filtering signal, and detect valid peaks in the electrocardiogram signal by using the peak occurrence time value.

In at least one variant, the valid peak detector may define a first time interval including a first average peak time value from among the average peak time values, calculate a first complexity value and a second complexity value in the first filtering signal and the second filtering signal, respectively, within the first time interval and, when both the first complexity value and the second complexity value exceed a preset threshold complexity level, determine, as a peak occurrence time value, the first average peak time value included in the first time interval.

In another variant, the preset threshold complexity level may be a value that is determined on the basis of complexity values of time intervals including R peaks designated in measured electrocardiogram signals.

In further another variant, the complexity values may be values that are calculated on the basis of an occurrence frequency of signals for each section of measured values.

In another variant, when a first window defined by a time interval does not include candidate peaks, the peak integrator may be implemented not to calculate an average peak time value corresponding to the first window.

According to one or more embodiments, a method of detecting signal peaks includes: sensing an electrocardiogram signal by using an electrode attached to the body; filtering the electrocardiogram signal into a first frequency band; filtering the electrocardiogram signal into a second frequency band; detecting first candidate peaks in a first filtering signal generated by the filtering and detecting second candidate peaks in a second filtering signal generated by the filtering; defining windows by time intervals and calculating, as average peak time values, average values of time values of the first candidate peaks and the second candidate peaks included in the windows; and calculating, in the first filtering signal and the second filtering signal, complexity values for time intervals including the average peak time values, determining a peak occurrence time value on the basis of the complexity value in the first filtering signal and the complexity value in the second filtering signal, and detecting valid peaks in the electrocardiogram signal by using the peak occurrence time value.

In at least one variant, the detecting of the valid peaks may include: defining a first time interval including a first average peak time value from among the average peak time values, calculating a first complexity value and a second complexity value in the first filtering signal and the second filtering signal, respectively, within the first time interval, and, when both the first complexity value and the second complexity value exceed a preset threshold complexity level, determining, as a peak occurrence time value, the first average peak time value included in the first time interval.

According to one or more embodiments, a computer program may be stored on a medium to perform, by using a computer, any one of methods according to one or more embodiments.

In addition, other methods and other systems for implementing one or more embodiments, and computer-readable recording media recording thereon a computer program for executing the method may be further included.

Other aspects, features, and advantages than those described above will become apparent from the following drawings, claims, and detailed description.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of a bio-signal measuring apparatus 100 according to one or more embodiments;

FIG. 2 is a block diagram of a bio-signal measuring apparatus 100′ according to one or more embodiments;

FIG. 3 is a flowchart of a method of detecting signal peaks according to one or more embodiments;

FIG. 4A is an example view illustrating a first filtering signal FD1;

FIG. 4B is an example view illustrating a second filtering signal FD2;

FIG. 4C is an example view illustrating a third filtering signal FD3;

FIG. 5A is an example view illustrating first candidate peaks CP1;

FIG. 5B is an example view illustrating second candidate peaks CP2;

FIG. 5C is an example view illustrating third candidate peaks CP3;

FIG. 6A is an exemplary diagram of candidate peaks detected according to one or more embodiments;

FIG. 6B is a view for explaining a process of determining an average peak time value, according to one or more embodiments;

FIG. 7A is a diagram of a signal filtered to a first frequency band according to one or more embodiments;

FIG. 7B is a diagram of a signal filtered to a second frequency band according to one or more embodiments;

FIG. 7C is a diagram of a signal filtered to a third frequency band according to one or more embodiments;

FIG. 8A is an example view illustrating an electrocardiogram signal; and

FIG. 8B is an example view illustrating an electrocardiogram signal from which signal peaks are detected, according to one or more embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the configuration and operation of the present disclosure will be described in detail with reference to embodiments of the present disclosure shown in the accompanying drawings.

While example embodiments are capable of various modifications and alternative forms, embodiments thereof are shown by way of example in the drawings and will herein be described in detail. The effects and features of the present disclosure and the accompanying methods thereof will become apparent from the following description of the embodiments, taken in conjunction with the accompanying drawings. However, the present disclosure is not limited to the embodiments described below, and may be embodied in various modes.

Hereinafter, embodiments of the present disclosure will be described in detail with reference to the accompanying drawings, and, when describing with reference to the drawings, the same or corresponding elements are given the same reference numerals and redundant descriptions thereof will be omitted.

In the following embodiments, the terms first, second, etc. may not be used in a limited sense but may be used to distinguish one element or component from another element or component.

The singular forms are intended to include the plural forms as well, unless the context clearly indicates otherwise.

The terms “comprises,” “includes,” “have,” etc. when used herein, specify the presence of stated features, elements, and/or components, but do not preclude the presence or addition of one or more other features, elements, or components.

In the drawings, elements or components may be exaggerated or reduced in sizes thereof for convenience of description. For example, the size and thickness of each element or component shown in the drawings are arbitrarily shown for convenience of description, so the present disclosure is not necessarily limited to what is shown.

When a certain embodiment is capable of being implemented differently, a particular process order may be performed differently from the described order. For example, two processes described in succession may be performed substantially simultaneously or may be performed in an order opposite to the described order.

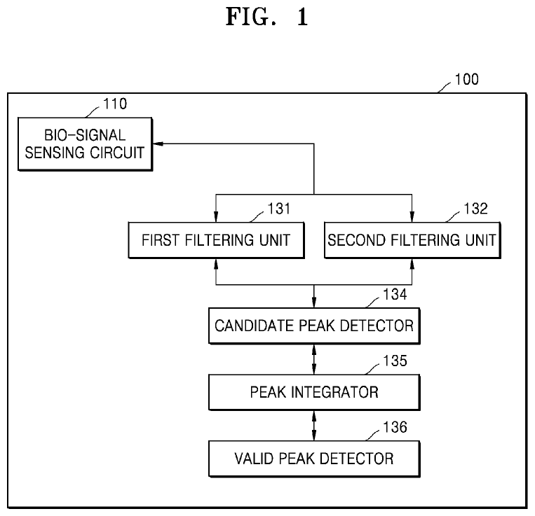

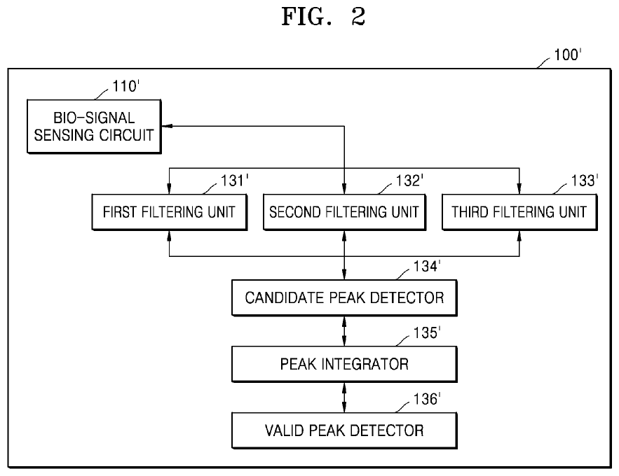

FIG. 1 is a block diagram of a bio-signal measuring apparatus 100 according to one or more embodiments. FIG. 2 is a block diagram of a bio-signal measuring apparatus 100′ according to one or more embodiments.

The bio-signal measuring apparatus 100 includes a bio-signal sensing circuit 110, a first filtering unit 131, a second filtering unit 132, a candidate peak detector 134, a peak integrator 135, and a valid peak detector 136. At least one of the first filtering unit 131, the second filtering unit 132, the candidate peak detector 134, the peak integrator 135, and the valid peak detector 136 may be implemented as a hardware component. At least one of the first filtering unit 131, the second filtering unit 132, the candidate peak detector 134, the peak integrator 135, and the valid peak detector 136 may be implemented as software code and executed by a separately provided processor. In some forms, the bio-signal sensing circuit 110, the first filtering unit 131, the second filtering unit 132, the candidate peak detector 134, the peak integrator 135, and the valid peak detector 136 may be provided and implemented in one apparatus, or in other forms, may be provided in two or more apparatuses.

The bio-signal measuring apparatus 100 may process a bio-signal sensed via the bio-signal sensing circuit 110. The bio-signal sensing circuit 110 may be invasively or non-invasively attached to the body of a user and sense a bio-signal of the user. The bio-signal sensing circuit 110 may acquire an electrocardiogram signal that is an electrical change according to a heartbeat obtained by an electrode. Although not shown in FIG. 1, the bio-signal sensing circuit 110 may include a measurement electrode for acquiring an electrocardiogram signal and an AD conversion unit for converting, into digital electrocardiogram data, an analog electrocardiogram signal measured by the measurement electrode. The bio-signal sensing circuit 110 may further include an amplification unit for amplifying an electrocardiogram signal.

The bio-signal measuring apparatus 100 may include the first filtering unit 131 and the second filtering unit 132 for filtering a bio-signal into determined frequency bands, and may include the candidate peak detector 134, the peak integrator 135, and the valid peak detector 136 for detecting peaks included in filtered signals.

An electrocardiogram signal is filtered into a first frequency band via the first filtering unit 131, and an electrocardiogram signal is filtered into a second frequency band via the second filtering unit 132. Here, for convenience of description, frequency conversion is used, but various types of conversion methods (e.g., wavelet conversion, Hilbert conversion, and the like) may be used.

The candidate peak detector 134 detects first candidate peaks included in a first filtering signal filtered by the first filtering unit 131. The candidate peak detector 134 detects second candidate peaks included in a second filtering signal filtered by the second filtering unit 132. The candidate peak detector 134 detects candidate peaks on the basis of QRS template.

The peak integrator 135 calculates average peak time values of the first candidate peaks of the first filtering signal and the second candidate peaks of the second filtering signal by using windows defined by time intervals. Here, a window may be set to have a width of a preset unit time. Here, the width (size) of the unit time may be determined on the basis of a heart rate of a user or an R-R interval.

The peak integrator 135 may merge the first candidate peaks with the second candidate peaks on the basis of time values and calculate, as an average peak time value, an average value of time values of the first candidate peaks and the second candidate peaks by using windows having determined sizes after the mergence. For example, when a first window includes a candidate peak A and a candidate peak B, an average value of a time value of the candidate peak A and a time value of the candidate peak B may be converted into an average peak time value. When the first window does not include candidate peaks, an average peak time value corresponding to the first window is recorded as nope and is not calculated.

In the above method, the peak integrator 135 may calculate average peak time values while moving, along the x axis, a window having a determined size in data including first candidate peaks and second candidate peaks.

The valid peak detector 136 calculates, in the first filtering signal and the second filtering signal, complexity values for time intervals including peak time values and detects a valid peak point on the basis of the complexity value in the first filtering signal and the complexity value in the second filtering signal. Here, a complexity value may be calculated by using a complexity calculation algorithm such as Shannon entropy or the like but is not limited thereto. The complexity value may be calculated in various methods such as Kolmogorov, monotone, prefix, and decision.

The complexity value may be determined on the basis of an occurrence frequency for each magnitude section of measured values of pieces of data of a measured bio-signal. Measured values of a signal may be divided into a plurality of magnitude bins on the basis of the magnitude thereof. Accordingly, a first occurrence frequency may be calculated by extracting data (points) of a signal included in a first magnitude bin. A high occurrence frequency in one magnitude bin may indicate that signals (data) are generated in a similar pattern, and a low occurrence frequency in each magnitude bin may indicate that signals have irregular patterns, i.e., complexity forms.

In other words, a complexity value may be calculated as in Equation 1 below by defining a magnitude bin generated on the basis of a value obtained by measuring a signal and calculating an occurrence frequency of the number of data points in each of magnitude bins.

$$SE = - {\sum }_{m = 1}^{M} p ( m ) {log}_{2} p ( m ) \qquad [ Equation 1 ]$$

wherein M is a bin magnitude value of the magnitude of a signal, and p(m) is an occurrence probability function of the signal in each bin interval. For example, when m is 1, p(m) may represent a ratio between the total number L of data points and the number N of data points corresponding to m=1.

$$p ( m ) = \frac{N}{L}$$

In another embodiment, a complexity value may be calculated by using a turning point ratio (TRR). In this case, when the number of turning points per unit time is great, a complexity value may be calculated high. When the number of turning points per unit time is less, a complexity value may be calculated to be low. A TRR may be a value for identifying a change in magnitude of two adjacent pieces of data at a particular point in time and representing a ratio of a turning point to the total length of data. Here, the turning point refers to a point at which the tangent of a signal is 0, i.e., a point at which a direction of a value of data is changed. A complexity value may be calculated as in Equation 2 below:

$$TPR = \frac{N ( ( {x}_{i} - {x}_{i - 1} ) ( {x}_{i} - {x}_{i + 1} ) > 0 )}{L} \qquad [ Equation 2 ]$$

wherein N (x) denotes the number of pieces of data satisfying condition x, and L denotes the magnitude of a data section. (N((xᵢ−xᵢ₋₁)(xᵢ−xᵢ₊₁)>0)) denotes the number of points (data) that is (xᵢ−xᵢ₋₁)(xᵢ−xᵢ₊₁)>0) in the section L, i.e., the number of turning points.

The valid peak detector 136 determines a first time interval including a first average peak time value, calculates a first complexity value of a first filtering for the first time interval, calculates a second complexity value of a second filtering signal for the first time interval, and, when the first complexity value and the second complexity value are detected to exceed a preset threshold complexity level, calculates a first peak time value as a peak occurrence time value. The valid peak detector 136 may detect, through the above process, peak occurrence time values in time intervals.

When at least one of the first complexity value and the second complexity value is detected not to exceed the preset threshold complexity level, the first peak time value does not become the peak occurrence time value.

The valid peak detector 136 may detect valid peaks in an electrocardiogram signal by using peak occurrence time values.

As used herein, a threshold complexity level that is a basis for selecting a peak occurrence time value may be determined through a process of performing machine learning by using, as input data, a complexity value of a time interval including peaks and using, as output data, whether or not a peak is valid but is not limited thereto. The threshold complexity level may be determined by using various types of neural networks, statistical techniques, or the like.

As shown in FIG. 2, peak occurrence time values may be detected by using signals filtered via a first filtering unit, a second filtering unit, and a third filtering unit to detect valid peaks. The bio-signal measuring apparatus 100′ of FIG. 2 may be implemented by including a bio-signal sensing circuit 110′, a first filtering unit 131′, a second filtering unit 132′, and a third filtering unit 133′. The bio-signal sensing circuit 110′ is the same as the bio-signal sensing circuit 110, and a detailed description thereof will be omitted herein.

At least one of the first filtering unit 131′, the second filtering unit 132′, the third filtering unit 133′, a candidate peak detector 134′, a peak integrator 135′, and a valid peak detector 136′ may be implemented as a hardware component. At least one of the first filtering unit 131′, the second filtering unit 132′, the third filtering unit 133′, the candidate peak detector 134′, the peak integrator 135′, and the valid peak detector 136′ may be implemented as software code and execute by a separately provided processor. The first filtering unit 131′, the second filtering unit 132′, the third filtering unit 133′, the candidate peak detector 134′, the peak integrator 135′, and the valid peak detector 136′ may be implemented in one apparatus or may be implemented in two or more apparatuses. The first filtering unit 131′, the second filtering unit 132′, the third filtering unit 133′, the candidate peak detector 134′, the peak integrator 135′, and the valid peak detector 136′ may be implemented as remote apparatuses to receive a bio signal from the bio-signal sensing circuit 110′ and process the bio-signal. The candidate peak detector 134′ detects first candidate peaks included in a first filtering signal filtered by the first filtering unit 131′, second candidate peaks included in a second filtering signal filtered by the second filtering unit 132′, and third candidate peaks included in a third filtering signal filtered by the third filtering unit 133′. The candidate peak detector 134′ may generate, as one integrated piece of data, the first candidate peaks, the second candidate peaks, and the third candidate peaks.

The peak integrator 135′ calculates average peak time values of the first candidate peaks of the first filtering signal, the second candidate peaks of the second filtering signal, and the third candidate peaks of the third filtering signal by using windows defined by time intervals. An average of time values of a 1-1^{th} candidate peak, a 1-2^{th} candidate peak, and a 1-3^{th} candidate peak included in a first window defined by a first time interval may be calculated and set as an average peak time value. The 1-1^{th} candidate peak, the 1-2^{th} candidate peak, and the 1-3^{th} candidate peak may belong to at least one of first candidate peaks, second candidate peaks, and third candidate peaks. The 1-1^{th} candidate peak, the 1-2^{th} candidate peak, and the 1-3^{th} candidate peak may be included in different filtering signals, respectively. The 1-1^{th} candidate peak and the 1-2^{th} candidate peak may be included in a fist filtering signal or the 1-1^{th} candidate peak, the 1-2^{th} candidate peak, and the 1-3^{th} candidate peak may be included in the first filtering signal.

The peak integrator 135′ may merge first candidate peaks, second candidate peaks, and third candidate peaks on the basis of time values and calculate, as an average peak time value, an average value of time values of the first candidate peaks, the second candidate peaks, and the third candidate peaks by using windows having determined time intervals (magnitudes) after the mergence. The number of candidate peaks included in a window may be 0 or may be three or more. For example, when a first window includes a candidate peak A, a candidate peak B, a candidate peak C, and a candidate peak D, an average value of time values of the candidate peaks A, B, C, and D may be converted into an average peak time value. When the first window does not include candidate peaks, an average peak time value corresponding to the first window is recorded as nope and is not calculated.

In the above method, the peak integrator 135′ may calculate average peak time values while moving, along the x axis, a window having a determined size in data including first candidate peaks and second candidate peaks.

The valid peak detector 136′ calculates, in first through third filtering signals, complexity values for time intervals including peak time values, respectively, and detects a peak occurrence time value on the basis of the respectively calculated complexity values. Here, a complexity value may be calculated by using a complexity calculation algorithm such as Shannon entropy, Sample entropy or Turning point ratio.

The valid peak detector 136′ determines a first time interval including a first average peak time value, calculates a first complexity value of a first filtering signal, a second complexity value of a second filtering signal, and a third complexity value of a third filtering signal for the first time interval, calculates a second complexity value of a second filtering signal for the first time interval, and, when the first complexity value and the second complexity value are detected to exceed a preset threshold complexity level, detects a first peak time value as a peak occurrence time value. The valid peak detector 136′ may detect valid peaks on the basis of peak occurrence time values calculated through the above process. When at least one of the first complexity value, the second complexity value, and the third complexity value is detected not to exceed the preset threshold complexity level, the first peak time value does not become the peak occurrence time value.

The valid peak detector 136′ may detect valid peaks in an electrocardiogram signal by using peak occurrence time values.

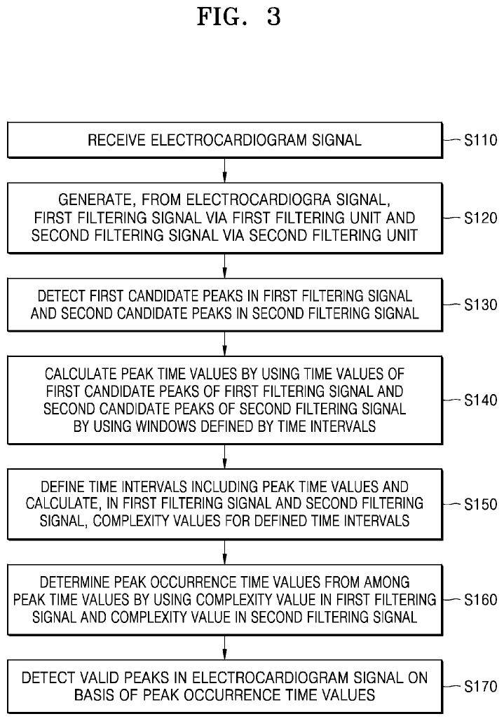

FIG. 3 is a flowchart of a method of detecting signal peaks according to one or more embodiments.

In operation S110, the bio-signal measuring apparatus 100 receives an electrocardiogram signal from a sensing circuit. In operation S120, the bio-signal measuring apparatus 100 filters the electrocardiogram signal into a first frequency band via the first filtering unit 131 and filters the electrocardiogram signal into a second frequency band via the second filtering unit 132.

In operation S130, the bio-signal measuring apparatus 100 detects first candidate peaks included in a first filtering signal filtered by the first filtering unit 131. The bio-signal measuring apparatus 100 detects second candidate peaks included in a second filtering signal filtered by the second filtering unit 132.

In operation S140, the bio-signal measuring apparatus 100 calculates average peak time values of the first candidate peaks of the first filtering signal and the second candidate peaks of the second filtering signal by using windows defined as time intervals. Here, a window may be set to have a width of a preset unit time. Here, the width (magnitude) of the unit time may be determined on the basis of a heartbeat of a user or an R-R interval.

The bio-signal measuring apparatus 100 may merge the first candidate peaks with the second candidate peaks on the basis of time values and calculate, as an average peak time value, an average value of time values of the first candidate peaks and the second candidate peaks by using windows having a determined size after the mergence. When a determined first window does not include candidate peaks, an average peak time value corresponding to the first window is recorded as nope and is not calculated.

The bio-signal measuring apparatus 100 may calculate average peak time values while moving, along the x axis, the window having the determined size in data including the first candidate peaks and the second candidate peaks.

In operation S150, the bio-signal measuring apparatus 100 calculates, in the first filtering signal and the second filtering signal, complexity values for time intervals including the peak time values and detects a valid peak point on the basis of the complexity value in the first filtering signal and the complexity value in the second filtering signal. Here, a complexity value may be calculated by using a complexity calculation algorithm such as Shannon entropy.

The complexity value may be determined on the basis of an occurrence frequency for each magnitude section of measured values of pieces of data of a measured bio-signal. Measured values of a signal may be divided into a plurality of magnitude bins on the basis of the magnitude thereof. Accordingly, a first occurrence frequency may be calculated by extracting pieces of data (points) of a signal included in a first magnitude bin. A high occurrence frequency in one magnitude bin may indicate that signals (data) are generated in a similar pattern, and a low occurrence frequency in each magnitude bin may indicate that signals have irregular patterns, i.e., complex forms.

In other words, a complexity value may be calculated as in Equation 1 below by defining magnitude bins generated on the basis of values obtained by measuring a signal and calculating an occurrence frequency of the number of data points in each of the magnitude bins.

In another embodiment, a complexity value may be calculated by using a turning point ratio (TPR). In this case, when the number of turning points per unit time is great, a complexity value may be calculated high. When the number of turning points per unit time is small, a complexity value may be calculated to be low. The TRR may be a value for identifying a change in magnitude of adjacent pieces of data at a particular point in time and representing a ratio of a turning point to the total length of data. Here, a turning point refers to a point at which the tangent of a signal is 0, i.e., a point at which a direction of a value of data is changed. A complexity value may be calculated as in Equation 2.

The bio-signal measuring apparatus 100 determines a first time interval including a first average peak time value, calculates a first complexity value of a first filtering signal for the first time interval, calculates a second complexity value of a second filtering signal for the first time interval, and, when the first complexity value and the second complexity value are detected to exceed a preset threshold complexity level, detects a first peak time value as a peak occurrence time value. In operation S160, the bio-signal measuring apparatus 100 may detect peak occurrence time values in time intervals through the above process.

In operation S170, the bio-signal measuring apparatus 100 may detect valid peaks in an electrocardiogram signal by using the peak occurrence time values.

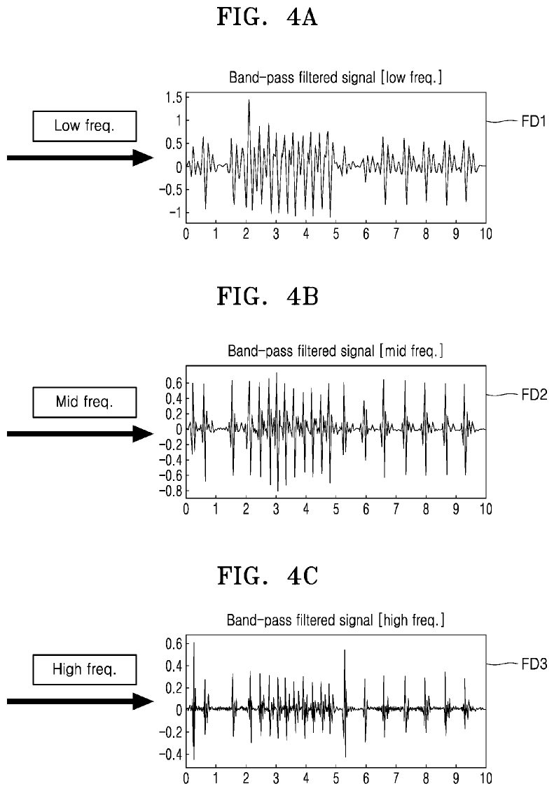

FIG. 4A is an example view illustrating a first filtering signal FD1, FIG. 4B is an example view illustrating a second filtering signal FD2, and FIG. 4C is an example view illustrating a third filtering signal FD3. The bio-signal measuring apparatus 100 may generate the first filtering signal FD1 by frequency-transforming with first frequency. The bio-signal measuring apparatus 100 may generate the second filtering signal FD2 by frequency-transforming with second frequency. The bio-signal measuring apparatus 100 may generate the third filtering signal FD3 by frequency-transforming with third frequency.

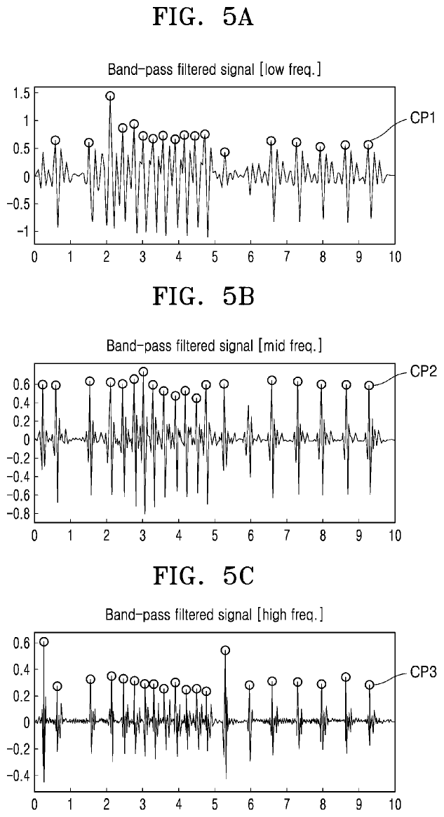

FIG. 5A is an example view illustrating first candidate peaks CP1, FIG. 5B is an example view illustrating second candidate peaks CP2, and FIG. 5C is an example view illustrating third candidate peaks CP3. The bio-signal measuring apparatus 100 may detect candidate peaks from first, second and third filtering signals. The bio-signal measuring apparatus 100 may detect first candidate peaks CP1 in the first filtering signal by using a form characteristic, a slope value, a threshold voltage value, an R-R interval, and the like of the first filtering signal. The bio-signal measuring apparatus 100 may detect second candidate peaks CP2 in the second filtering signal by using a form characteristic, a slope value, a threshold voltage value, an R-R interval, and the like of the second filtering signal. The bio-signal measuring apparatus 100 may detect third candidate peaks CP3 in the third filtering signal by using a form characteristic, a slope value, a threshold voltage value, an R-R interval, and the like of the third filtering signal.

The bio-signal measuring apparatus 100 may detect candidate peaks in a filtering signal by using a form characteristic, a slope value, a threshold voltage value, an R-R interval, and the like of the filtering signal.

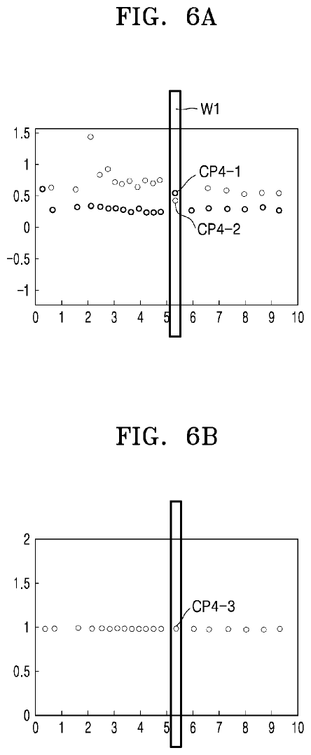

FIG. 6A is an exemplary diagram of candidate peaks detected according to one or more embodiments. FIG. 6B is a view for explaining a process of determining an average peak time value, according to one or more embodiments.

As shown in FIG. 6A, in data including first candidate peaks CP1, second candidate peaks CP2, and third candidate peaks CP3, one or more candidate peaks CP4-1 and CP4-2 included in a window W1 defined by a certain time interval may be extracted, and an average of time values of the candidate peaks CP4-1 and CP4-2 may be calculated as an average peak time value. As shown in FIG. 6B, a point corresponding to the average peak time value calculated from the candidate peaks CP4-1 and CP4-2 may be converted into CP4-3.

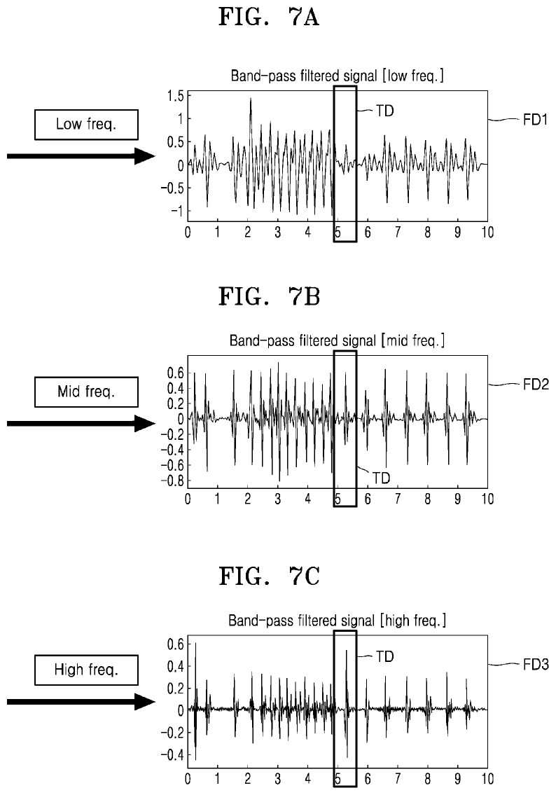

FIG. 7A is a diagram of a signal filtered to a first frequency band according to one or more embodiments. FIG. 7B is a diagram of a signal filtered to a second frequency band according to one or more embodiments. FIG. 7C is a diagram of a signal filtered to a third frequency band according to one or more embodiments. As shown in FIG. 7A, FIG. 7B, and FIG. 7C, a time interval TD including a time value of CP4-3 may be defined, and complexity values for the defined time interval TD may be calculated from first through third filtering signals, respectively.

When all of the complexity values for the time interval TD exceed a preset threshold complexity level, the time value of CP4-3 included in the time interval TD may be determined as a peak occurrence time value.

Accordingly, according to one or more embodiments, the bio-signal measuring apparatus 100 may determine peak occurrence time values of peak points in an electrocardiogram signal.

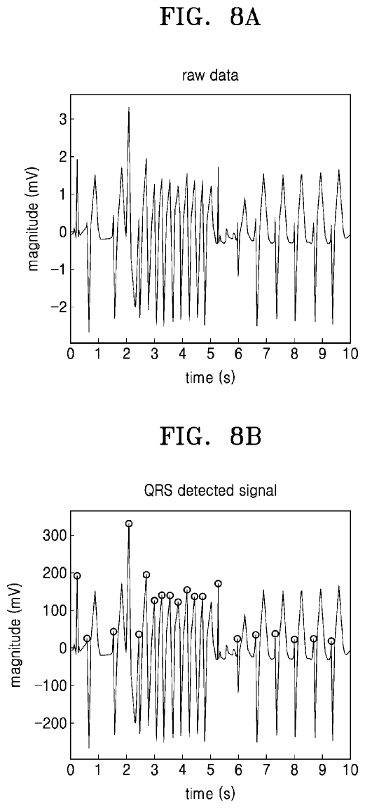

FIG. 8A is an example view illustrating an electrocardiogram signal and FIG. 8B is an example view illustrating an electrocardiogram signal from which signal peaks are detected, according to one or more embodiments.

As shown in FIG. 8A and FIG. 8B, the measured electrocardiogram signal may be converted into data including the detected peak data.

The apparatus described above may be embodied as hardware components, software components, and/or a combination of hardware components and software components. For example, the apparatus and components described in the embodiments may be embodied by using one or more general purpose computers or special purpose computers such as a processor, a controller, an arithmetic logic unit (ALU), a digital signal processor, a microcomputer, a field programmable gate array (FPGA), a programmable logic unit (PLU), a microprocessor, or any other apparatuses capable of executing and responding to instructions. A processing apparatus may execute an operating system (OS) and one or more software applications executed on the OS. In addition, the processing apparatus may access, store, manipulate, process, and generate data in response to the execution of software. For convenience of description, one processing apparatus may be described as being used, but one of ordinary skill in the art may understand that the processing apparatus may include a plurality of processing elements and/or a plurality of types of processing elements. For example, the processing apparatus may include a plurality of processors or one processor and one controller. Also, the processing apparatus may include another processing configuration such as a parallel processor.

Software may include computer program, code, instructions, or a combination of one or more thereof, and configure the processing apparatus to operate as wanted or independently or collectively instruct the processing apparatus. Software and/or data may be permanently or temporarily embodied in any type of machine, a component, a physical device, virtual equipment, a computer storage medium or device, or transmitted signal waves to be interpreted by the processing apparatus or to provide the processing apparatus with instructions or data. The software may be distributed over networked computer systems to be stored or executed in a distributed manner. The software and data may be stored in one or more computer-readable recording media.

The method according to the embodiments may be embodied in the form of program instructions that may be executed through various types of computer means and then recorded in a computer-readable recording medium. The computer-readable recording medium may include program instructions, data files, data structures, and the like alone or in combination. The program instructions recorded on the computer-readable recording medium may be particularly designed and configured for the embodiments or may be well known to and used by one of ordinary skill in the computer software art. Examples of the computer-readable recording medium include magnetic media such as a hard disk, a floppy disk, and a magnetic tape, optical media such as a CD-ROM and a DVD, magneto-optical media such as a floptical disk, and a hardware device particularly configured to store and execute program instructions such as ROM, RAM, flash memory, and the like. Examples of the program instructions include not only machine language code generated by a compiler, but also high-level language code that may be executed by a computer by using an interpreter or the like. The hardware device described above may be configured to operate as one or more software modules so as to perform operations of the embodiments, and the reverse thereof is the same.

According to one or more embodiments, signal peaks may be detected by using a complexity value of a signal around a peak.

In addition, according to one or more embodiments, signal peaks in a time interval having a complexity value exceeding a determined threshold complexity level may be detected via machine learning or an artificial intelligence algorithm.

Although the embodiments have been described above by limited embodiments and drawings, various modifications and changes may be made from the above description by one of ordinary skill in the art. For example, the described techniques may be performed in a different order than the described method, and/or components of the described system, structure, device, circuit, and the like may be combined or joined in a different form than the described method, or even if replaced or substituted by other components or equivalents, and an appropriate result may be achieved.

Therefore, other embodiments, other aspects, and equivalents to claims also belong to the scope of claims that will be described below.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A bio-signal measuring apparatus for detecting signal peaks, the bio-signal measuring apparatus comprising:
a bio-signal sensing circuit configured to sense an electrocardiogram signal by using an electrode attached to the body;
a first filtering unit configured to filter the electrocardiogram signal into a first frequency band;
a second filtering unit configured to filter the electrocardiogram signal into a second frequency band;
a candidate peak detector configured to detect first candidate peaks in a first filtering signal filtered by the first filtering unit and detect second candidate peaks in a second filtering signal filtered by the second filtering unit;
a peak integrator configured to define windows by time intervals and calculate, as average peak time values, average values of time values of the first candidate peaks and the second candidate peaks included in the windows; and
a valid peak detector configured to:
calculate, in the first filtering signal and the second filtering signal, complexity values for time intervals including the average peak time values,
determine a peak occurrence time value on the basis of the complexity value in the first filtering signal and the complexity value in the second filtering signal, and
detect valid peaks in the electrocardiogram signal by using the peak occurrence time value.

2. The bio-signal measuring apparatus of claim 1, wherein the candidate peak detector detects on the basis of QRS template.

3. The bio-signal measuring apparatus of claim 1, wherein the complexity value is from one of Shannon entropy, Sample entropy, and Turning point ratio.

4. The bio-signal measuring apparatus of claim 1, wherein the valid peak detector:
defines a first time interval including a first average peak time value from among the average peak time values;
calculates a first complexity value and a second complexity value in the first filtering signal and the second filtering signal, respectively, within the first time interval; and,
when both the first complexity value and the second complexity value exceed a preset threshold complexity level, determines, as a peak occurrence time value, the first average peak time value included in the first time interval.

5. The bio-signal measuring apparatus of claim 4, wherein the preset threshold complexity level corresponds to a value that is determined on the basis of complexity values of time intervals including R peaks designated in measured electrocardiogram signals.

6. The bio-signal measuring apparatus of claim 1, wherein the complexity values are values that are calculated on the basis of an occurrence frequency of signals for each section of measured values.

7. The bio-signal measuring apparatus of claim 1, wherein, when a first window defined by a time interval does not include candidate peaks, the peak integrator is implemented not to calculate an average peak time value corresponding to the first window.

8. A method of detecting signal peaks, the method comprising:
sensing an electrocardiogram signal by using an electrode attached to the body;
filtering the electrocardiogram signal into a first frequency band;
filtering the electrocardiogram signal into a second frequency band;
detecting first candidate peaks in a first filtering signal generated by the filtering and detecting second candidate peaks in a second filtering signal generated by the filtering;
defining windows by time intervals and calculating, as average peak time values, average values of time values of the first candidate peaks and the second candidate peaks included in the windows;
calculating, in the first filtering signal and the second filtering signal, complexity values for time intervals including the average peak time values;
determining a peak occurrence time value on the basis of the complexity value in the first filtering signal and the complexity value in the second filtering signal; and
detecting valid peaks in the electrocardiogram signal by using the peak occurrence time value.

9. The method of claim 8, wherein detecting the first candidate peaks further comprises detecting the first candidate peaks on the basis of QRS template; and
detecting the second candidate peaks further comprises detecting the second candidate peaks on the basis of QRS template.

10. The method of claim 8, wherein calculating the complexity values further comprises calculating the complexity value from one of Shannon entropy, Sample entropy, Turning point ratio.

11. The method of claim 8, wherein detecting of the valid peaks further includes:
defining a first time interval including a first average peak time value from among the average peak time values;
calculating a first complexity value and a second complexity value in the first filtering signal and the second filtering signal, respectively, within the first time interval; and
when both the first complexity value and the second complexity value exceed a preset threshold complexity level, determining, as a peak occurrence time value, the first average peak time value included in the first time interval.

12. The method of claim 11, further comprising determining the preset threshold complexity level on the basis of complexity values of time intervals including R peaks designated in measured electrocardiogram signals.

13. The method of claim 8, wherein calculating the complexity values further comprises calculating the complexity values as values that are calculated on the basis of an occurrence frequency of signals for each section of measured values.

14. The method of claim 8, wherein calculating the average peak time values further includes:
when a first window defined by a time interval does not include candidate peaks, not calculating an average peak time value corresponding to the first window.

15. A computer program stored on a computer-readable storage medium to perform, by using a computer, the method of claim 8.
